(19) United States
(12) Patent Application Publication
(10) Pub. No.: US 2026/0013985 A1
(43) Pub. Date: Jan. 15, 2026

(54) PROSTHETIC VALVE ASSEMBLIES WITH EXPANSION CONTROL RODS

(71) Applicant: EDWARDS LIFESCIENCES CORPORATION, Irvine, CA (US)

(72) Inventors: Tamir S. Levi, Zikhron Yaakov (IL); Noam Nir, Pardes-Hanna (IL)

(73) Assignee: EDWARDS LIFESCIENCES CORPORATION, Irvine, CA (US)

(21) Appl. No.: 19/332,875

(22) Filed: Sep. 18, 2025

Related U.S. Application Data
(63) Continuation of application No. PCT/US2024/022312, filed on Mar. 29, 2024.
(60) Provisional application No. 63/455,904, filed on Mar. 30, 2023.

(51) Int. Cl. A61F 2/24

(52) U.S. Cl. CPC A61F 2/2418; A61F 2/2436; A61F 2/2439; A61F 2220/0075

(57) ABSTRACT

The present disclosure relates to prosthetic valves that can be expanded to selected maximal diameters. In an example, a prosthetic valve assembly comprises a prosthetic valve having an annular frame, one or more expansion control rods disposed radially outward to the frame, and one or more flexible tension members extending circumferentially around the frame, and comprising one or more local loops wrapped around the corresponding expansion control rods. The flexible tension member is configured to radially expand to a first diameter when all local loops are wrapped around the corresponding expansion control rods, and expand to a second diameter greater than the first diameter, when one of the expansion control rod is retracted from the flexible tension member, thereby allowing the corresponding local loop to unravel.

### CROSS-REFERENCE TO RELATED APPLICATIONS

This application is a continuation of International Application No. PCT/US2024/022312, filed Mar. 29, 2024, which claims the benefit of U.S. Provisional Application No. 63/455,904, filed Mar. 30, 2023, which is incorporated by reference herein.

### FIELD

The present disclosure relates to prosthetic valves and assemblies that include expansion control rods coupled to flexible tension members extending around the frame of the valve, configured to control radial expansion of prosthetic valve, as well as to methods of utilization thereof.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic valve is mounted in a crimped state on the distal end of a delivery apparatus and advanced through the patient's vasculature (e.g., through a femoral artery and the aorta) until the prosthetic valve reaches the implantation site in the heart. The prosthetic valve is then radially expanded to its functional size, for example, by deploying the prosthetic valve from the delivery device so that the prosthetic valve can self-expand. Once the prosthetic valve is fully expanded, it is then operational inside of the patient's heart and replaces the function of the native heart valve for the useful life of the prosthetic valve.

Most self-expandable, transcatheter valves comprise a radially expandable and compressible annular metal frame and prosthetic leaflets mounted inside the frame. The frame can comprise a plurality of circumferentially extending rows of angled struts defining rows of open cells of the frame. The prosthetic valve can include an outer sealing member (also referred to as an “outer skirt”) affixed to an outer surface of the frame for sealing the prosthetic valve against tissue of the native heart valve. The outer skirt typically is attached to frame via sutures. During implantation, a self-expandable heart valve can be disposed within a sheath or a capsule of the delivery device that retains the valve in its crimped state during transcatheter delivery, and retraction of the sheath can allow the self-expandable heart valve to be released from the sheath and expand to its functional size.

### SUMMARY

Prosthetic valves that rely on balloon inflation for expansion can be referred to as “balloon-expandable” prosthetic valves. Similarly, prosthetic valves that rely on a mechanical actuator for expansion can be referred to as “mechanically expandable” prosthetic valves. Mechanically expandable prosthetic heart valves and balloon-expandable heart valves can provide one or more advantages over self-expandable prosthetic heart valves. For example, after positioning at the desired implantation site, expansion of mechanically expandable prosthetic valves and balloon expandable valves can be selectively controlled to cause the valve to expand to various desired diameters and/or to achieve gradual expansion of the prosthetic valve. However, despite these benefits, mechanically expandable prosthetic heart valves and balloon-expandable valves can have one or more disadvantages relative to self-expandable valves, such as requiring complex actuator systems, which can increase the cost of the delivery assembly and the complexity of the implantation procedure. Accordingly, a self-expandable prosthetic valve having a mechanism that can facilitate controllable radial expansion thereof is desired.

Described herein are examples of prosthetic valves including a self-expandable annular frame comprising a plurality of interconnected struts. The prosthetic valve can optionally further include one or more expansion control rods through which a flexible tension member is looped, configured to enable gradual or step-wise expansion of the prosthetic valve. Additionally, described herein are delivery apparatus configured for use with the prosthetic valves, as well as associated methods.

In one of its basic configurations, a prosthetic valve assembly comprises a prosthetic valve, at least one expansion control rod disposed radially outward to a frame of the prosthetic valve, and at least one flexible tension member extending circumferentially around the frame. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the frame of the prosthetic valve can optionally be an annular frame movable between a radially compressed and a radially expanded configuration, the frame defining a central longitudinal axis.

In some examples, the at least one expansion control rod can optionally extend between a rod proximal end portion and a rod distal end portion in parallel to the central longitudinal axis.

In some examples, the at least one flexible tension member can comprise at least one local loop wrapped around the at least one expansion control rod.

In some examples, the at least one flexible tension member is optionally configured to radially expand to a first diameter when the at least one local loop is wrapped around the at least one expansion control rod, and expand to a second diameter greater than the first diameter, when the at least one expansion control rod is retracted from the at least one flexible tension member, thereby optionally allowing the corresponding at least one local loop to unravel.

In one of its basic methods, a method of assembling a prosthetic valve assembly comprises providing a prosthetic valve that includes a frame movable between a radially compressed and a radially expanded configuration, in a radially expanded configuration of the frame. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the method comprises positioning at least one expansion control rod radially outward of the frame.

In some examples, the method comprises extending at least one flexible tension member around the frame.

In some examples, the method optionally comprises forming at least one local loop by wrapping the at least one flexible tension member around the at least one expansion control rod.

In one of its basic methods, a method comprises selecting a maximum diameter for a prosthetic valve assembly based at least in part on a patient's native anatomy. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the prosthetic valve assembly optionally comprises a prosthetic valve comprising a prosthetic valve.

In some examples, the prosthetic valve optionally comprises an annular frame movable between a radially compressed and a radially expanded configuration.

In some examples, the prosthetic valve assembly optionally comprises at least one expansion control rod disposed radially outward to the frame.

In some examples, the prosthetic valve assembly optionally comprises at least one flexible tension member extending circumferentially around the frame.

In some examples, the at least one expansion control rod is optionally coupled to the at least one flexible tension member via a local loop of the at least one flexible tension member wrapped around the at least one expansion control rod.

In some examples, the method comprises retracting any of the at least one expansion control rod from the at least one flexible tension member in a manner that unravels the corresponding local loop, so as to allow the at least one flexible tension member to expand to the maximum diameter.

In some examples, the method comprises crimping the prosthetic valve to the radially compressed configuration of the frame.

In some examples, the method optionally comprises inserting the crimped prosthetic valve into a capsule of a delivery apparatus.

In some examples, the method optionally comprises advancing the delivery assembly to a selected implantation site inside the body of the patient.

In some examples, the method optionally comprises deploying the prosthetic valve assembly out of the capsule, thereby allowing it to expand to the selected maximum diameter.

In one of its basic configurations, a delivery assembly comprising a prosthetic valve assembly and a delivery apparatus comprising at least one pull-assembly. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the prosthetic valve assembly can comprise a prosthetic valve.

In some examples, the prosthetic valve optionally comprises an annular frame movable between a radially compressed and a radially expanded configuration and defining a central longitudinal axis.

In some examples, the prosthetic valve assembly can comprise at least one expansion control rod disposed radially outward to the frame.

In some examples, the prosthetic valve assembly can comprise at least one flexible tension member extending circumferentially around the frame.

In some examples, the at least one expansion control rod can optionally extend between a rod proximal end portion and a rod distal end portion in parallel to the central longitudinal axis.

In some examples, the at least one flexible tension member can comprise at least one local loop wrapped around the at least one expansion control rod.

In some examples, the delivery apparatus can comprise a releasable pull member releasably coupled to the at least one expansion control rod.

In some examples, the delivery apparatus can comprise an outer sleeve disposed around, and optionally axially movable relative to, the releasable pull member.

In some examples, the at least one pull-assembly is optionally configured to release the at least one expansion control rod from the at least one flexible tension member by axially pulling it when coupled thereto.

In some examples, the at least one flexible tension member is optionally configured to radially expand to a first diameter when the at least one local loop is wrapped around the at least one expansion control rod, and expand to a second diameter greater than the first diameter, when the at least one expansion control rod is retracted from the at least one flexible tension member by the at least one pull-assembly, thereby optionally allowing the corresponding at least one local loop to unravel.

The innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1A is a perspective view of an exemplary prosthetic valve.

FIG. 1B is a perspective view of a frame of the prosthetic valve of FIG. 1A.

FIGS. 2A-2C shows different expansion states of an exemplary prosthetic valve assembly.

FIG. 3 shows an exemplary prosthetic valve assembly comprising expansion control rods coupled to two axially spaced flexible tension members.

FIG. 4A shows an exemplary frame expanded to a V-shaped profile.

FIG. 4B shows an exemplary frame expanded to an A-shaped profile

FIG. 5 shows an exemplary prosthetic valve assembly comprising a first series of expansion control rods coupled to a proximal flexible tension member, and a second series of expansion control rods coupled to a distal flexible tension member.

FIG. 6 shows an exemplary prosthetic valve assembly comprising expansion control rods having non-uniform diameters along their lengths.

FIG. 7 shows an exemplary prosthetic valve assembly comprising expansion control rods equipped with wider rod heads.

FIG. 8A shows an exemplary pull-assembly coupled to an expansion control rod.

FIG. 8B shows flexible elongated members of the pull-assembly of FIG. 8A disengaged from, yet still in contact with, the rod head of the expansion control rod.

FIG. 8C shows the pull-assembly of FIG. 8B retracted from the expansion control rod.

FIGS. 9A-9B show a distal portion of an exemplary delivery assembly in different states of engagement with a prosthetic valve.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a prosthetic valve and/or a delivery apparatus. In general, the end of any device, apparatus or assembly closest to the user of the apparatus (such as during an implantation procedure) is the proximal end, and the end farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

The terms “coupled” and “coupling” are used herein according to their broad and ordinary meanings. For example, where a first feature, element, component, device, or member, is described as being “coupled” with or to a second feature, element, component, device, or member, such description may be understood as indicating that the first feature, element, component, device, or member, or portion thereof, is physically/mechanically attached, fixed, fastened, mounted, connected, linked, or joined to, or united, associated together, or integrated with, or embedded at least partially within, or otherwise physically related to, the second feature, element, component, device, or member, or portion thereof, whether directly or indirectly. A “coupling” can refer to any device, structure, form, tool, mechanism, means, position, apparatus, or portion, component, or position thereof that at least partially facilitates and/or effects/achieves the coupling of two or more features, elements, components, devices, or members, and/or portions thereof.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first,” “second,” and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a,” “b,” “c,” etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

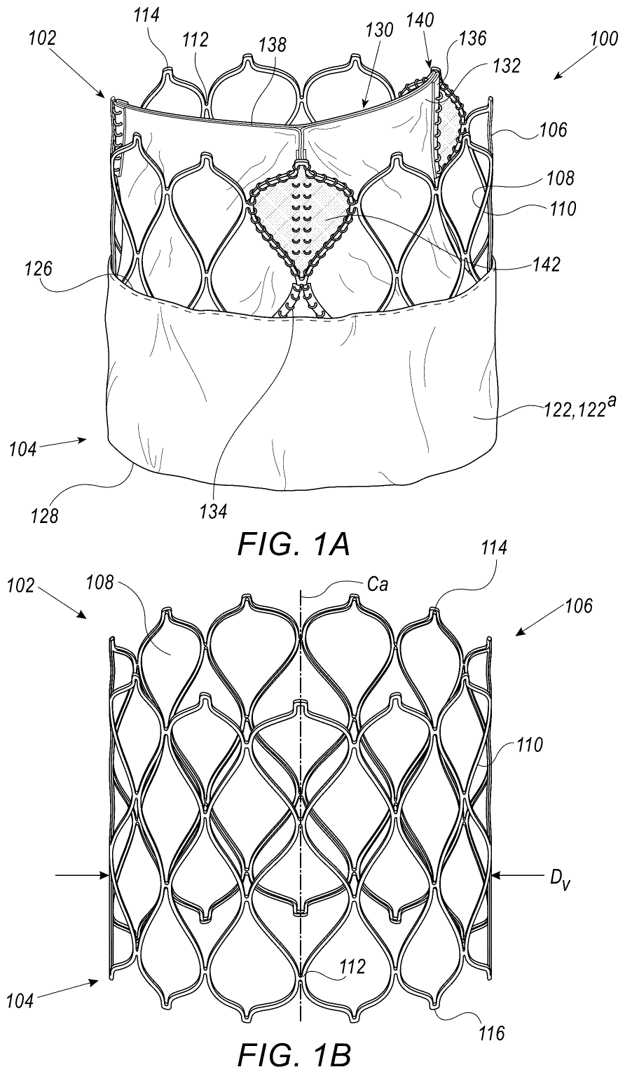

Reference is now made to FIGS. 1A-1B, showing an exemplary prosthetic valve 100, including various components thereof. The prosthetic valve 100 is deliverable to a patient's target site over a catheter (not shown), and is radially expandable and compressible between a radially compressed, or crimped, state, and a radially expanded state (expanded state of the valve 100 is illustrated in FIGS. 1A-1B). The expanded state may include a range of diameters to which the valve 100 may expand, between the compressed state and a maximal diameter reached at a fully expanded state. Thus, a plurality of partially expanded states may relate to any expansion diameter between radially compressed or crimped state, and maximally expanded state. It should be understood that the disclosed prosthetic valves, valve assemblies and delivery assemblies, can be adapted for use of valve replacement in any of the native annuluses of the heart (e.g., the aortic, pulmonary, mitral, and tricuspid annuluses), and can be used with any of various delivery approaches (e.g., retrograde, antegrade, transseptal, transventricular, transatrial, etc.).

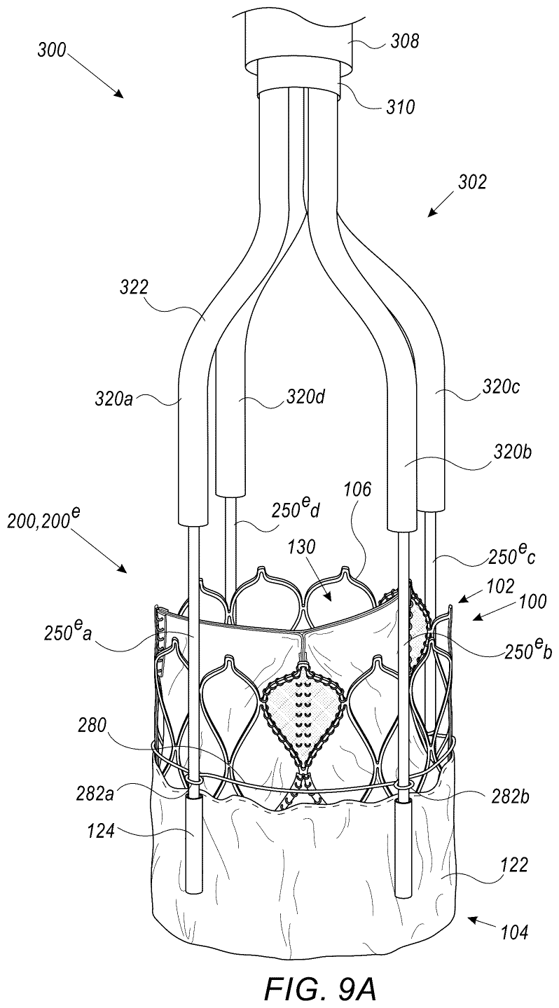

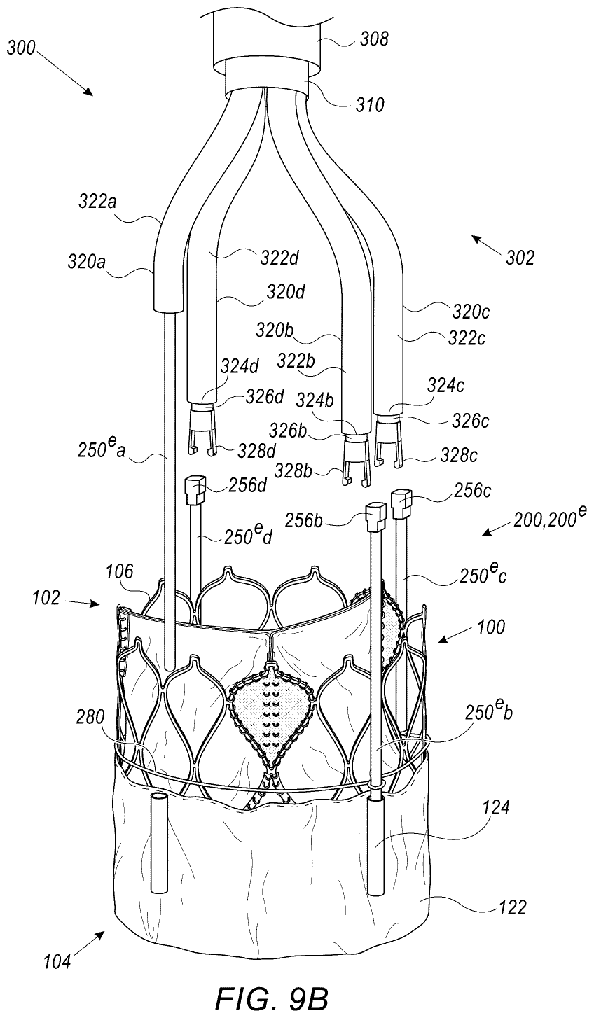

As stated above, a prosthetic valve 100 can optionally be delivered to the site of implantation via a delivery assembly 300 (an example of which is described below respect to FIGS. 9A-9B) carrying the valve 100 in a radially compressed or crimped state, toward the target site, to be mounted against the native anatomy, by expanding the valve 100 via various expansion mechanisms. FIGS. 1A-1B show an example of a self-expandable valve 100. Self-expandable valves include a frame that is shape-set to automatically expand as soon as an outer retaining structure, such as a capsule or a portion of a shaft, is withdrawn proximally relative to the prosthetic valve.

Other types of valves may include other expansion mechanisms, such as mechanical expansion mechanisms or balloon inflatable valves (not shown). Mechanically expandable valves are a category of prosthetic valves that rely on a mechanical actuation mechanism for expansion. The mechanical actuation mechanism usually includes a plurality of expansions and locking assemblies, releasably coupled to respective actuation assemblies of a delivery apparatus, controlled via a handle for actuating the actuation assemblies to expand the prosthetic valve to a desired diameter. The expansions and locking assemblies may optionally lock the valve's position to prevent undesired recompression thereof, and disconnection of the actuation assemblies from the expansions and locking assemblies, to enable retrieval of the delivery apparatus once the prosthetic valve is properly positioned at the desired site of implantation.

Balloon expandable valves generally involve a procedure of inflating a balloon within a prosthetic valve, thereby expanding the prosthetic valve within the desired implantation site. Once the valve is sufficiently expanded, the balloon is deflated and retrieved along with the delivery apparatus. It is to be understood that a prosthetic valve can be expanded by a combination of such mechanisms. For example, a mechanically expandable valve can be also formed as a partially self-expandable valve, such that once released from an outer retaining structure as described above, the valve can self-expand to a diameter that is larger than the crimped diameter, but is less than the fully expanded diameter, such that mechanical actuation can be employed to expand the valve further as required.

A prosthetic valve 100 can comprise an inflow end 104, an outflow end 102, and a central longitudinal axis Ca extending in a direction from the inflow end 104 to the outflow end 102. In some instances, the outflow end 102 is the distal end of the prosthetic valve 100, and the inflow end 104 is the proximal end of the prosthetic valve 100. Alternatively, depending for example on the delivery approach of the valve, the outflow end can be the proximal end of the prosthetic valve, and the inflow end can be the distal end of the prosthetic valve.

The term “outflow”, as used herein, refers to a region of the prosthetic valve through which the blood flows through and out of the valve 100.

The term “inflow”, as used herein, refers to a region of the prosthetic valve through which the blood flows into the valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The valve 100 comprises an annular frame 106 movable between a radially compressed configuration and a radially expanded configuration, and a valvular structure 130 mounted within the frame 106. The frame 106 can optionally be made of various suitable materials, including plastically-deformable materials such as, but not limited to, stainless steel, a nickel-based alloy (e.g., a cobalt-chromium or a nickel-cobalt-chromium alloy such as MP35N alloy), polymers, or combinations thereof. When constructed of a shape-memory materials such as, but not limited to, nickel-titanium alloy (e.g., Nitinol), the frame can be crimped to a radially compressed state and restrained in the compressed state by insertion into a shaft or equivalent mechanism of a delivery apparatus (not shown).

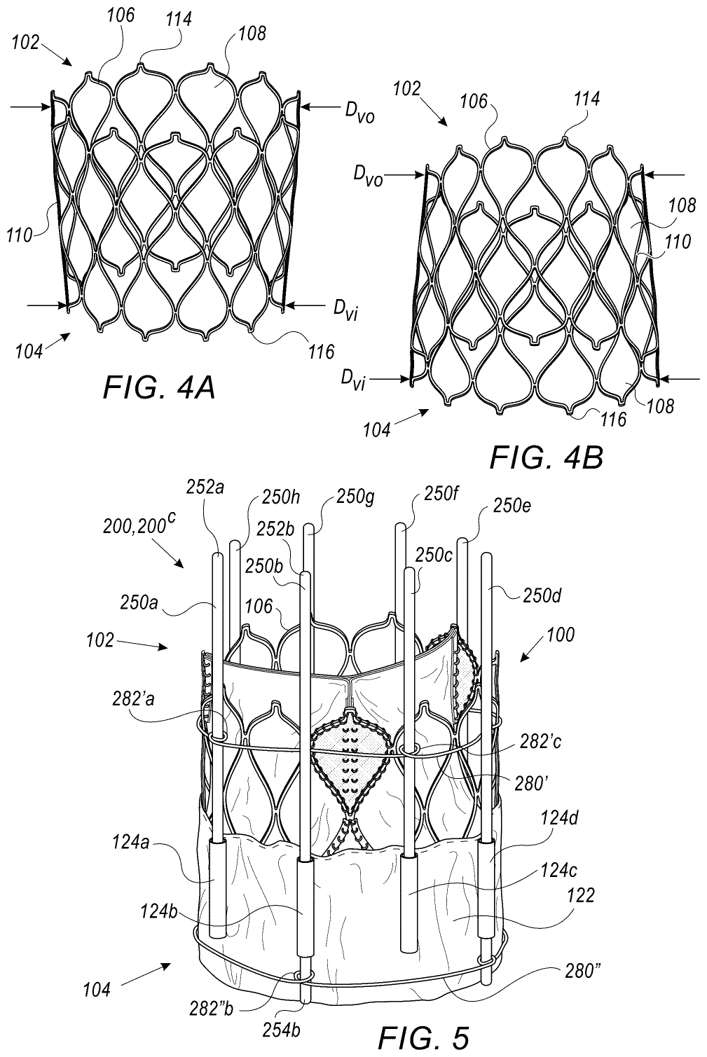

In the example illustrated in FIGS. 1A-1B, the frame 106 is an annular, stent-like structure comprising a plurality of intersecting struts 110 arranged in a lattice-type pattern. The frame 106 can optionally have one or more rows of openings or cells 108 defined by intersecting struts, such as the angled struts 110 shown in FIGS. 1A-1B. The struts 110 can optionally intersect at junctions 112, as well as at outflow apices 114 at the outflow end 102, and inflow apices 116 at the inflow end 104. The frame 106 can optionally have a cylindrical or substantially cylindrical shape having an expanded diameter Dᵥ that can be constant from the inflow end 104 to the outflow end 102 of the frame as shown in FIG. 1B, or the frame can vary in diameter Dᵥ along the height of the frame, as shown for example in FIGS. 4A-4B and/or as disclosed in U.S. Pat. No. 9,155,619, which is incorporated herein by reference.

The struts may optionally be pivotable or bendable relative to each other, so as to permit frame expansion or compression. In some implementations, the frame 106 can optionally be formed from a single piece of material, such as a metal tube, via various processes such as, but not limited to, laser cutting, electroforming, and/or physical vapor deposition, while retaining the ability to collapse/expand radially.

The valvular structure 130 comprises a plurality of leaflets 132 (e.g., three leaflets), positioned at least partially within the frame 106, and configured to regulate flow of blood through the prosthetic valve 100 from the inflow end 104 to the outflow end 102. While three leaflets 132 arranged to collapse in a tricuspid arrangement, are shown in the exemplary embodiment illustrated in FIG. 1A, it will be clear that a prosthetic valve 100 can include any other number of leaflets 132. The lower edge of valvular structure 130 desirably has an undulating, curved scalloped shape. By forming the leaflets with this scalloped geometry, stresses on the leaflets are reduced, which in turn improves durability of the valve. Moreover, by virtue of the scalloped shape, folds and ripples at the belly of each leaflet (the central region of each leaflet), which can cause early calcification in those areas, can be eliminated or at least minimized. The scalloped geometry also reduces the amount of tissue material used to form valvular structure 130, thereby allowing a smaller, more even crimped profile at the inflow end of the valve.

The leaflets 132 can optionally be made, in whole or part, from biological material (e.g., pericardium), bio-compatible synthetic materials, or other such materials. Further details regarding transcatheter prosthetic valves, including the manner in which leaflets 132 can be coupled to the frame 106 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

In some examples, the prosthetic valve 100 can optionally comprise at least one skirt or sealing member. An exemplary outer skirt 122 that can be mounted on the outer surface of the frame 106 is illustrated in FIG. 1A, configure to function, for example, as a sealing member retained between the frame 106 and the surrounding tissue of the native annulus against which the prosthetic valve 100 is mounted, thereby reducing risk of paravalvular leakage past the prosthetic valve 100. The outer skirt 122 can optionally be connected to the frame 106 using suitable techniques or mechanisms. For example, the outer skirt 122 can optionally be sutured to the frame 106 via sutures that can extend around the struts 110. The outer skirt 122 extends between a skirt inflow end 128 and a skirt outflow end 126. In the illustrated example, the outer skirt's inflow and outflow ends 128, 126 are shown to be relatively linear around the circumference of the frame, yet it is to be understood that any of the skirt inflow end 128 and/or skirt outflow end 126 can be curved, zig-zagged, or have any suitable shape or combination of shapes. Moreover, while the skirt inflow end 128 is shown to be aligned with the inflow end 104 of the frame 106, it is to be understood that in other implementations, the skirt inflow end 128 can be proximally offset from the inflow end 104.

In some examples, the prosthetic valve can optionally comprise an inner skirt instead of and/or in addition to the outer skirt 122. Such an inner skirt can be similarly configured to function, for example, as a sealing member to prevent or decrease perivalvular leakage. An inner skirt can further function as an anchoring region for leaflets 132 to the frame 106, and/or function to protect the leaflets 132 against damage which may be caused by contact with the frame 106, for example during valve crimping or during working cycles of the prosthetic valve 100. An inner skirt can optionally be disposed around and attached to the inner surface of frame 106, while the leaflets can be sutured to the inner skirt along a scalloped line. An inner skirt can be coupled to the frame 106 via sutures or another form of coupler.

Any of the inner skirt and/or outer skirt 122 can be made of various suitable biocompatible materials, such as, but not limited to, various synthetic materials (e.g., PET) or natural tissue (e.g. pericardial tissue). In some cases, the inner skirt can optionally be formed of a single sheet of material that extends continuously around the inner surface of frame 107. In some cases, the outer skirt 122 can optionally be formed of a single sheet of material that extends continuously around the outer surface of frame 106. While some of the cells 108 can be covered by the inner skirt and/or the outer skirt, at least a portion of the cells 108 can optionally remain uncovered, such as cells 108 which are closer to the outflow end 102 of the prosthetic valve.

The inflow or cusp edges 134 of the leaflets 132 can be secured to the frame 106 directly or indirectly, such as by being sutured directly to the frame, being sutured to an inner skirt, and/or via one or more connecting skirts. Further examples and methods of attaching skirts and seal members to a frame, as well as method and techniques for coupling leaflets 132 to the frame 106, with or without connecting skirts, are disclosed in US Pat. Publication No. 2018/0028310, which is incorporated herein by reference.

Adjacent leaflets 132 can optionally be arranged together to form prosthetic valve commissures 140 that are coupled (directly or indirectly) to respective portions of the frame 106, thereby securing an upper portion (e.g., above the scalloped line) of the valvular structure to the frame 106. In some examples, each leaflet 132 can optionally comprise opposing tabs 136. Each tab 136 can optionally be secured to an adjacent tab 136 of an adjacent leaflet 132 to form a commissure 140 that is secured to the frame 106. The tabs 136 can be folded in various manners, for example to form radially extending layers and circumferentially extending layers facing the frame. Radially extending layers can extends radially inward from a location on the frame 106 to free edge 138, also termed coaptation edges, of the leaflet.

During valve cycling, the leaflets 132 can articulate at the inner most edges of the tab layers, which helps space the leaflets away from the frame 106 during normal operation of the prosthetic valve. This is particular advantageous in cases where the prosthetic valve 100 is not fully expanded to its nominal size when implanted in a patient. As such, the prosthetic valve 100 can be implanted in a wider range of patient annulus sizes.

Optionally, the prosthetic valve 100 can further comprise a plurality of support members 142 that can be made of a relatively flexible and soft materials, including synthetic materials (e.g., PET fabric) or natural tissue (e.g. bovine pericardium), attached to struts 110 of cells 108. The number of support members 142 can optionally match the number of commissures 140, wherein each commissure 140 can be mounted to the frame 106 by being attached (e.g., sutured) to a corresponding support member 142, as shows in the example illustrated in FIG. 1A. A commissure 140 can optionally be formed by folding the tabs 136 and stitching them to each other, and/or to additional components of the commissure, such as reinforcement members, fabrics and the like, according to various configurations disclosed in US Pat. Publication No. 2018/0028310, which is incorporated herein by reference.

As described above, self-expandable prosthetic valves have advantages over mechanically-expandable and balloon-expandable valves in that they do not require a separate mechanical actuator system to transition from a crimped state to a radially expanded state. For example, after transcatheter delivery of a conventional self-expandable prosthetic valve in a crimped state to a patient's native heart valve, a sheath of the delivery apparatus can be partially retracted and (initially) a first end portion of the prosthetic valve (e.g., an inflow end portion) is released from the sheath and can be partially expanded. As the sheath is further retracted, an intermediate portion of the prosthetic valve is released and partially expanded, while the first end portion of the prosthetic valve is moved into a further expanded state (e.g., an almost fully expanded state or a fully expanded state). Finally, a second end portion of the prosthetic valve (e.g., an outflow end portion) is released from the sheath as the sheath is further retracted, thereby resulting in full radial expansion of all portions of the prosthetic valve and disengagement of the prosthetic valve from the delivery apparatus.

Although expansion can be controlled at least in part via controlling an extent of retraction of the sheath, conventional self-expandable prosthetic heart valves lack a mechanism for precisely controlling a degree of radial expansion and/or enabling gradual radial expansion of the prosthetic valve. Furthermore, conventional self-expandable prosthetic heart valves lack a mechanism for enabling concurrent radial expansion of multiple or all portions of the valve (e.g., concurrent radial expansion of the inflow end, the intermediate portion, and the outflow end), and lack a mechanism for independently controlling radial expansion of various portions of the valve, for example if the valve needs to be expanded to a tapering profile having different diameters of the inflow end and the outflow end.

Described herein are examples of prosthetic valve assemblies 200 configured for transcatheter implantation. A prosthetic valve assembly 200 can optionally include a prosthetic valve 100 according to any example described above, having a frame 106 that is a self-expandable frame, and at least one expansion control rod 250 coupled to at least one flexible tension member 280 extending circumferentially around the frame 106. The self-expandable frame 106 is biased toward a radially expanded configuration of the prosthetic valve 100, but can optionally be selectively radially compressed or radially expanded to transition the prosthetic valve 100 between the compressed configuration and the expanded configuration. In other examples, the frame 106 can be a partially self-expandable frame that is configured to radially expand the prosthetic valve to a partially expanded state. In such examples, the prosthetic valve 100 can optionally include an additional mechanical actuation mechanism for transitioning the prosthetic valve to the fully radially expanded state.

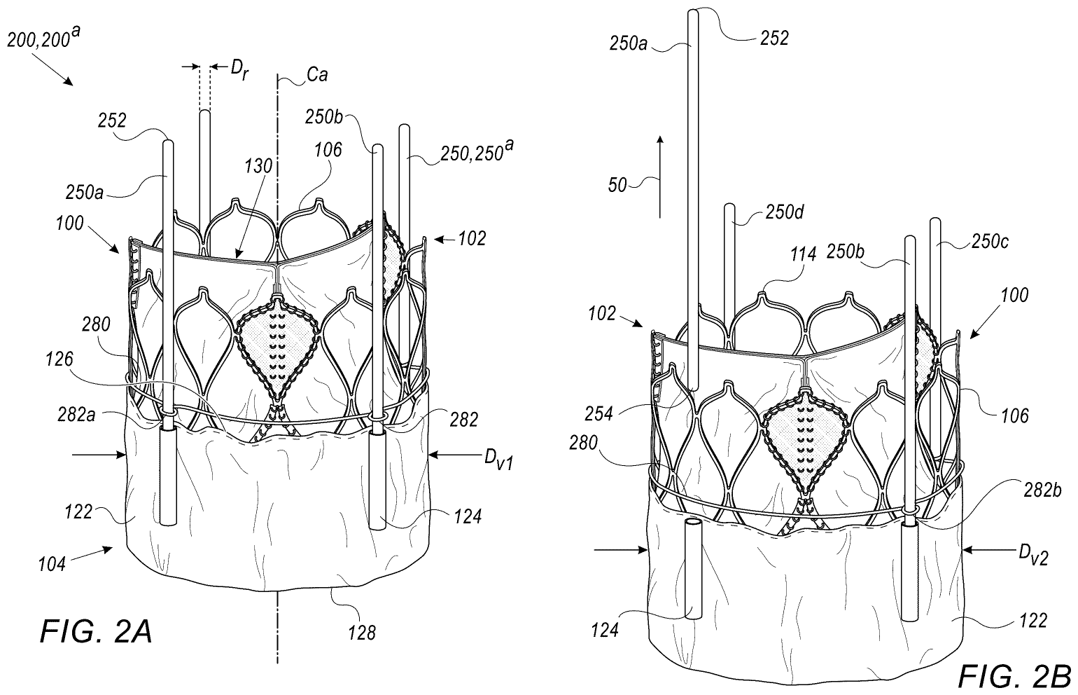

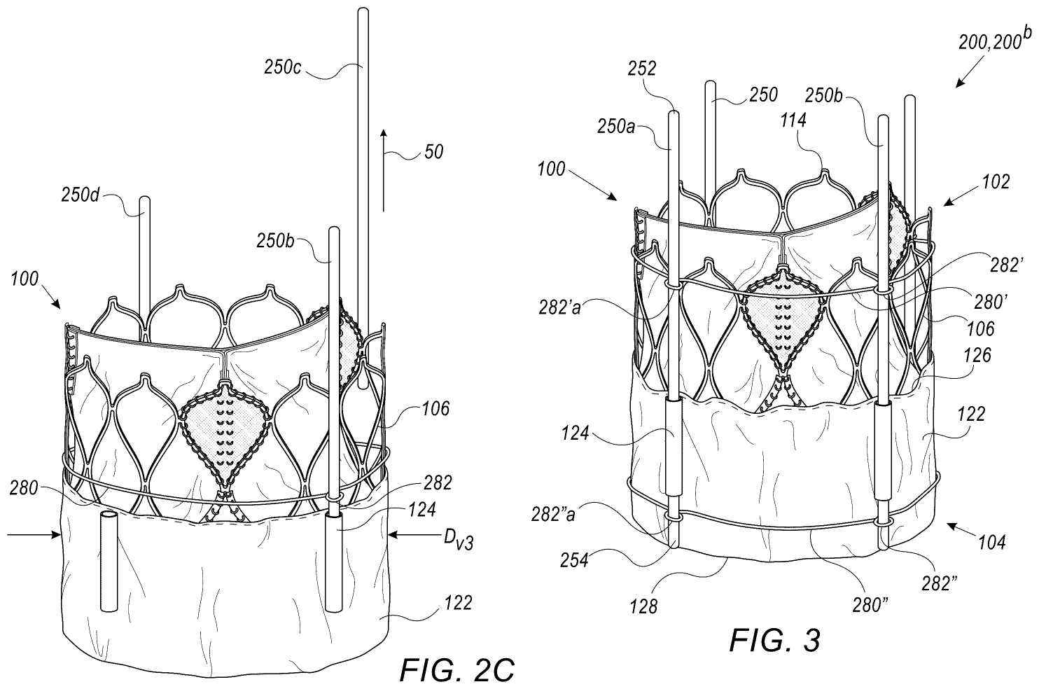

FIGS. 2A-2C show the transition between optional states an exemplary prosthetic valve assembly 200 can assume, for example prior to implantation within the patient's body. One or more expansion control rods 250 with one or more flexible tension member 280 can be optionally assembled on prosthetic valve 100 in a radially expanded configuration thereof, as shown in FIG. 2A. Prior to insertion into the patient's body, a crimping device (not shown) can be optionally used to crimp the prosthetic valve 100 to the compressed configuration, which can be then inserted into a patient's body over a delivery apparatus or stored in this configuration up to utilization thereof for implantation into the patient's body.

As illustrated, a prosthetic valve assembly 200 comprises at least one flexible tension member 280, that can optionally be in the form of a string or suture, looped around at least a portion of the circumference of the frame 106, and optionally around the entire circumference of the frame 106. At least one expansion control rod 250 is disposed on the outer side of frame 106, axially extending parallel to the central longitudinal axis Ca between rod proximal end portion 252 and rod distal end portion 254, such that the flexible tension member 280 forms at least one local loop 282 around the corresponding expansion control rod 250.

The expansion control rod 250 is assembled to the prosthetic valve 100 such that a portion of the rod 250 extends between the inflow 104 and outflow 102 ends of the frame 106, having the rod distal end portion 254 positioned distal to the outflow end 102, while another portion of the rod 250 can optionally extend proximally from the frame 106, such that the rod proximal end portion 252 can be positioned proximal to the outflow end 102. The at least one flexible tension member 280 is disposed around the frame 106 between the inflow 104 and outflow 102 ends, such that the corresponding one or more local loop(s) 282 can be optionally wrapped around the portion of the corresponding rod(s) 250 extending distally from the outflow end 102.

As mentioned, the prosthetic valve 100 can be configured to radially self-expand from a radially compressed state to a partially or fully expanded state under resilient and/or clastic forces of the frame 106. The flexible tension member 280 can optionally be utilized to retain the frame 106 in a compressed or partially expanded diameter, while increasing the diameter of the flexible tension member 280 can allow the frame 106 to expand further, potentially up to a fully expanded or functional diameter. Otherwise stated, the flexible tension member 280 can apply a radially inwardly directed force to the frame 106 that can optionally be gradually lessened such that the frame 106 can expand at a controlled rate (e.g., a rate selected by the physician) to a selected diameter.

Each rod 250 is configured to be releasably coupled to the flexible tension member 280 and/or prosthetic valve 100, by a local loop 282 of the flexible tension member 280 formed around the rod 250 while the remainder of the flexible tension member 280 is disposed around the frame 106 in an expanded or partially expanded state thereof, such that the tension applied by the flexible tension member 280 around the frame 106 and by the local loop(s) 282 around the rod(s) 250 is sufficient to prevent spontaneous displacement or detachment of the rod(S) 250.

The flexible tension member 280 serves as a restraining mechanism, and can comprise a wire, string, suture, and/or cable. As mentioned, the flexible tension member 280 extends circumferentially around the frame 106, and can optionally be further coupled to the one or more expansion control rod(s) 250 via local loop(s) 282. In other examples, the flexible tension member 280 can optionally be disposed entirely around the frame 106, as illustrated. In some examples, the flexible tension member 280 can optionally be coupled to the frame 106 via one or more sutures (not illustrated). In some examples, the flexible tension member 280 can optionally be weaved through cells 108 of the frame 106, for example by weaving it in an in-and-out pattern along struts 110 (weaving pattern not illustrated). In some examples, the frame 106 can optionally comprise a plurality of additional cyclets or apertures (not shown) through which portions of the flexible tension member 280 can be threaded.

The flexible tension member 280 can optionally extend at least partially around the circumference of the frame 106. In the example illustrated in FIGS. 2A-2C, the flexible tension member 280 extends around frame 106 such that it forms a single loop encircling the frame 106. In other examples, the flexible tension member 280 can optionally be disposed such that it spans a distance less than the full circumference of the frame 106, for example by being tied at end portions thereof to struts 110 of the frame 106. In some cases, the flexible tension member 280 can optionally be attached to the frame 106 at one or more locations, for example by forming knots around struts of the frame 106 at one or two ends of the flexible tension member 280.

Any of the prosthetic valves 100 and/or prosthetic valve assemblies 200 disclosed herein can optionally be adapted to be implanted in the native aortic annulus, as well as in the other native annuluses of the heart (the pulmonary, mitral, and tricuspid valves). The disclosed prosthetic valves 100 and/or prosthetic valve assemblies 200 also can optionally be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can optionally be implanted within a previously implanted prosthetic valve (which can be a prosthetic surgical valve or a prosthetic transcatheter heart valve) in a valve-in-valve procedure.

In some examples, the disclosed prosthetic valves 100 and/or prosthetic valve assemblies 200 can optionally be implanted within a docking or anchoring device that is implanted within a native heart valve or a vessel. For example, the disclosed prosthetic valves can optionally be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can optionally be implanted within a docking device implanted within or at the native mitral valve, such as disclosed in PCT Publication No. WO2020/247907, which is incorporated herein by reference. In another example, the disclosed prosthetic valves can optionally be implanted within a docking device implanted within the superior or inferior vena cava for replacing the function of a diseased tricuspid valve, such as disclosed in U.S. Publication No. 2019/0000615, which is incorporated herein by reference.

Various exemplary implementations for prosthetic valve assemblies 200 and/or components thereof can be referred to, throughout the specification, with superscripts, for case of explanation of features that refer to such exemplary implementations. It is to be understood, however, that any reference to structural or functional features of any assembly, apparatus or component, without a superscript, refers to these features being commonly shared by all specific exemplary implementations that can be also indicated by superscripts. In contrast, features emphasized with respect to an exemplary implementation of any assembly, apparatus or component, including prosthetic valve assembly 200, referred to with a superscript, may be optionally shared by some but not necessarily all other exemplary implementations. For example, prosthetic valve assembly 200^{a} is an exemplary implementation of prosthetic valve assembly 200, and thus includes all of the features described for prosthetic valve assembly 200 throughout the current disclosure, except that while a prosthetic valve assembly 200 can be generally provided with any number of flexible tension members 280, prosthetic valve assembly 200^{a} includes a single flexible tension member 280, as will be described in further detail below.

Optionally, the at least one expansion control rod 250 can comprise a plurality of expansion control rods 250. A plurality of expansion control rods 250 can optionally be utilized, in some implementations, to enable gradual or step-wise radial expansion of prosthetic valve 100, or to choose a desired maximal expansion diameter from a variety of discrete optional expansion diameters. The flexible tension member 280 can optionally have a total circumferential length corresponding to a diameter of the annular frame 106 when in the radially expanded configuration. When a plurality of expansion control rods 250 are assembled on the valve 100, they can be evenly or unevenly spaced from each other around the circumference of the frame 106.

FIGS. 2A-2C show various states of an exemplary prosthetic valve assembly 200^{a}. As mentioned above, prosthetic valve assembly 200^{a} is an exemplary implementation of prosthetic valve assembly 200, and thus includes all of the features described for prosthetic valve assembly 200 throughout the current disclosure, except that while prosthetic valve assembly 200 can include any number of flexible tension members 280, prosthetic valve assembly 200^{a} includes a single flexible tension member 280 that can be disposed around a central portion of the frame 106 (between the inflow end 104 and the outflow end 102).

Optionally, each expansion control rod 250 can be formed as an elongated cylindrical member that can be rigid along its length, and can optionally have a uniform diameter along its length, as shown for expansion control rods 250^{a} in FIGS. 2A-2C. Each expansion control rod 250 is axially movable relative to prosthetic valve 100, such that it can optionally be retracted or removed away from the prosthetic valve 100. In some examples, each expansion control rod 250 can optionally be proximally pulled away from prosthetic valve 100 and from flexible tension member 280, in a manner that releases it from the local loop 282 and from prosthetic valve 100.

In some examples, the prosthetic valve 100 further comprises means that stabilize and align the one or more expansion control rod 250 in position around the frame 106 when assembled to each other, but allow axial movement of the one or more expansion control rod 250 to allow it to be released from the valve 100. In some examples, the outer skirt 122 further comprises one or more sleeves 124, that can optionally be axially extending along at least a portion of the height of the skirt 122, between the skirt outflow end 126 and the skirt inflow end 128. The number of sleeves 124 can optionally match the number of expansion control rods 250, wherein each sleeve 124 is optionally configured to accommodate at least a portion of a corresponding expansion control rod 250 therein, and allow axial movement of the corresponding expansion control rod 250 therethrough, optionally out of it in a released state of the expansion control rod 250.

When provided, the sleeves 124 can be equally or unequally disposed around the circumference of the outer skirt 122, and can optionally be attached (e.g., sutured) to the outer skirt 122 so as to extend radially away from the base layer of outer skirt 122, away from the central longitudinal axis Ca. During assembly, the rods 250 can optionally be inserted into sleeves 124, such that the position of the sleeves 124 dictates the desired circumferential position of the rods 250 around the frame 106.

In some examples, each sleeve 124 can optionally have the height of the outer skirt 122, extending from the skirt outflow end 126 and the skirt inflow end 128. In some examples, each sleeve 124 can optionally be shorter than the height of the outer skirt 122, such as by extending from the skirt outflow end 126 but terminating proximal to the skirt inflow end 128, as illustrated. In some examples, each sleeve 124 can optionally be longer than the height of the outer skirt 122, such as by further extending proximally from the skirt outflow end 126 and/or further extending distally from the skirt inflow end 128. In some examples, each sleeve 124 can optionally be open ended at one end thereof and closed on the opposite end, such as having an open proximal end through which a rod 250 can be inserted, and a distal closed end that can prevent the rod distal end portion 254 from passing farther distally from the sleeve 124. In some examples, the sleeve 124 can optionally be open ended at both ends thereof, allowing free passage of the rod 250 therethrough.

While outer skirt 122 is shown to include sleeves 124 throughout the Figures, it is to be understood that this is shown by way of illustration and not limitation, and that in some examples, prosthetic valve 100 and/or an outer skirt 122 thereof may optionally be devoid of sleeves 124. In some examples, the prosthetic valve 100 can optionally be devoid of additional means that stabilize and align the one or more expansion control rod 250. For examples, it may be sufficient for the local loops 282 through which control rods 250 pass, to provide adequate support to the rods 250 while being looped therearound. In some examples, other means for aligning and/or retaining rods 250 can be optionally provided, instead of or in addition to sleeves 124 of outer skirt 122. For example, prosthetic valve 100 can optionally include eyelets, tubes, and/or suture loops (not shown) coupled to the frame 106 or other components of the prosthetic valve 100, allowing similar axial movement of the rod(s) 250 therethrough.

Each flexible tension member 280 is configured to expand to a maximum diameter, which can optionally be the maximum diameter selected for prosthetic valve Dᵥ, such that the flexible tension member 280 allows expansion of the frame 106 to the maximum diameter Dᵥ, and prevents further expansion of the frame 106 past the maximum selected diameter Dᵥ.

In the example illustrated in FIGS. 2A-2C, four expansion control rods 250a, 250b, 250c and 250d are shown, all four of which are coupled to the frame 106 and reside in four corresponding sleeves 124 in FIG. 2A. It is to be understood that four expansion control rods 250 are shown by way of example, and that any prosthetic valve assembly 200 disclosed herein can optionally include any other number of expansion control rods 250, such as a single expansion control rod 250, two, three, or more than four expansion control rods 250. Any reference to a plurality of expansion control rods 250 throughout the current specification can similarly refer to a single expansion control rod 250, unless stated otherwise. The terms “expansion control rod 250” and “rod 250”, as used herein, are interchangeable.

When expansion control rods 250 are assembled on prosthetic valve 100, local loops 282 of the flexible tension member 280 are formed around the respective rods 250, thereby tensioning the flexible tension member 280 and shortening the lengths of the adjustable loop sections circumferentially extending between adjacent rods 250, in a manner that will reduce the diameter defined by the rods 250 and prevent expansion of the frame 106 beyond the diameter defined by flexible tension member 280.

As shown in the illustrated example of prosthetic valve assembly 200^{a}, the flexible tension member 280 can optionally be positioned approximately halfway between the inflow end 104 and the outflow end 102 of the prosthetic valve 100 so that the restraining force of the flexible tension member 280, when restricting the frame 106 to a partially expanded diameter.

As mentioned above, a prosthetic valve 100 can optionally be configured for expansion within a range of working diameters. For example, a prosthetic valve 100 can optionally have a self-expandable frame 106 designed to be fully expanded to a diameter of 30 mm. in an unrestricted state thereof. Such a prosthetic valve can optionally have a valvular structure 130 designed to properly function (e.g., by having the leaflets 132 sufficiently coapting) within a range of 26-30 mm., in which case partial expansion of the frame 106 to a working diameter within this range can be desired to fit into a specific anatomy of the patient, or for implantation within a previously implanted prosthetic valve. The prosthetic valve assembly 200ª illustrated in FIGS. 2A-2C can be representative of such an exemplary case. For example, when the flexible tension member 280 does not include any local loops 282 looped around any rod 250, the frame 106 can be allowed to expand to a maximal working diameter of 30 mm., while each local loop 282 can optionally compress the diameter of the flexible tension member 280 by about 1 mm.

In such an example, the diameter Dᵣ of each rod 250, at least at the region of local loop 282, can optionally be set such that the circumferential perimeter of the local loop, which is the diameter Dᵣ multiplied by x, is equal to the desired length of 1 mm., such that when four local loops 282 are formed around four rods 250 as shown in FIG. 2A, the frame 106 can be expanded up to a partial expansion diameter Dᵥ₁ of 26 mm, which can be also referred to as a first diameter.

If the expansion diameter of the valve 100 should not exceed this value of Dᵥ₁, all four rods 250 can remain in position, as shown in FIG. 2A. FIG. 2B shows a state of the valve 100 allowed to expand to a greater partially expanded diameter Dᵥ₂ of about 27 mm. (which can be referred to as a second diameter) by withdrawing one of the rods 250, such as rod 250a shown in FIG. 2B to be pulled in a proximal direction 50 away from prosthetic valve 100. Withdrawal of the rod 250a releases the rod from the valve 100 and the flexible tension member 280 disposed therearound, thereby freeing the corresponding portion of local loop 282a such that the diameter of the flexible tension member 280 disposed around the frame 106 is enlarged by the corresponding length of the unraveled loop 282a.

FIG. 2C shows another state of the prosthetic valve 100, in which one additional rod 250c is retracted and released from the flexible tension member 280, for example by being pulled in a proximal direction 50 in the illustrated example, such that the frame is allowed to further expand to a diameter of 28 mm. (which can be referred to as a third diameter) in this state. While not explicitly illustrated, another rod 250 can optionally be released to allow expansion to a diameter of 29 mm., and the final rod can optionally be released to allow expansion to a maximal diameter of 30 mm. if desired.

The number of rods 250 and local loops 282 formed therearound can optionally be chosen according to the number of discrete diameters desired within a working range of the prosthetic valve, and the diameter of the rods 250 and local loops 282 can optionally be chosen according to the desired values to partial expansion diameters within the working range.

Release of an appropriate number of expansion control rods 250 can optionally be performed prior to an implantation procedure, for example to fit the valve 100 in a specific anatomical size of a patient's annulus or an inner diameter of another device into which the prosthetic valve 100 is to be implanted. After removal of the appropriate number of rods 250 (if removal of any rods 250 is required), the prosthetic valve 100 and/or prosthetic valve assembly 200 can optionally be crimped and inserted into a sheath and/or capsule of a delivery apparatus, configured to retain the prosthetic valve 100 in a compressed configuration during delivery toward the site of implantation.

During an implantation procedure, the prosthetic valve 100 can optionally be advanced through the patient's vasculature in this crimped or compressed state thereof. Once the valve 100 is positioned at the target implantation site (e.g., the aortic annulus in the case of aortic valve replacement), the sheath and/or capsule can optionally be retracted, allowing the prosthetic valve to self-expand up to the diameter dictated by the flexible tension member 280 (such as any of the states shown in FIGS. 2A-2C), so as to mount it against the surrounding tissue. Once the valve is fully expanded, the delivery apparatus can optionally be retrieved from the patient's body, leaving the prosthetic valve in place.

FIG. 3 shows an exemplary prosthetic valve assembly 200^{b}. Prosthetic valve assembly 200^{b} is an exemplary implementation of prosthetic valve assembly 200, and thus includes all of the features described for prosthetic valve assembly 200 throughout the current disclosure, except that the at least one expansion control rod 250 is coupled to two flexible tension members 280, forming two local loops 282′ and 282″ axially spaced from each other. In the illustrated example, prosthetic valve assembly 200^{b} includes a proximal flexible tension members 280′, positioned closer to outflow end 102, and a distal flexible tension member 280″, positioned closer to inflow end 104.

In the illustrated example, the sleeves 124 are shown to be formed as tubular sleeves, open-ended on both sides, such that the rods 250 can optionally extend distally beyond the distal end of sleeves 124. In the illustrated configuration, each of a plurality of rods 250 is shown to be coupled to the valve 100 by a proximal local loop 282′ and a distal local loop 282″, such that retraction of the rod 250 can optionally free the corresponding sections of both proximal and distal flexible tension members 280′, 280″, so as to evenly expand both the proximal and distal portion of the frame 106.

It is to be understood that while a single flexible tension member 280 is illustrated in FIGS. 2A-2C, and two flexible tension members 280 are illustrated in FIG. 3, a prosthetic valve assembly 200 can optionally include any other number of flexible tension members 280, such as three or more members 280, which can be equally or unequally axially spaced from each other.

While a cylindrical frame 106 is illustrated in FIG. 1B, having a relatively uniform valve diameter Dᵥ between the inflow end 104 and outflow end 102 in the expanded configuration, in some implementations it may be desirable to deploy the frame to a non-cylindrical shape. The deployed shape of the prosthetic valve 100 is important because it can affect how the prosthetic valve anchors or interfaces with the native valve annulus, the proportions of the prosthetic valve that are located in one chamber versus the other chamber across the native valve, and/or the location(s) where the prosthetic valve engages the native tissue. The shape of the expanded prosthetic valve 100 can also affect various hemodynamic parameters of the prosthetic valve, such as the pressure drop across the prosthetic valve, the orifice area at the inflow and outflow, and the degree to which the leaflets 132 open and close during valve operation.

Thus, in some cases, it may be desirable to deploy the prosthetic valve 100 to a non-cylindrical shape, such as a Y-shape, an hourglass shape, a V-shape, an A-shape or frustoconical shape, and the like. FIG. 4A shows an exemplary frame 106 expanded to a V-shaped profile, such that the frame diameter Dᵥₒ at the outflow end 102 is greater than the frame diameter Dᵥᵢ at the inflow end 104. FIG. 4B shows an exemplary frame 106 expanded to an A-shaped profile, such that the frame diameter Dᵥᵢ at the inflow end 104 is greater than the frame diameter Dᵥₒ at the outflow end 102.

In use, a physician may analyze the patient's specific anatomy (e.g., using angiograms and/or CT-scans) prior to the implantation procedure, and can select an appropriate maximum diameter Dᵥᵢ for the inflow end 104 and/or the maximum diameter Dᵥₒ for the outflow end 102 of the frame. The physician can optionally remove any expansion control rods 250 that restrict the expansion diameter of the corresponding flexible tension member(s) 280 to a size that is less than that of the selected maximum diameter at the appropriate region of the frame. Accordingly, once the prosthetic valve assembly 200 has been delivered to the implantation site using a delivery apparatus, and an optional capsule in which the prosthetic valve assembly 200 was optionally retained during delivery, has been optionally retracted, the inflow and/or outflow ends of the prosthetic valve 100 can optionally self-expand to a diameter not greater than the selected maximum diameter(s).

FIG. 5 shows an exemplary prosthetic valve assembly 200^{c}. Prosthetic valve assembly 200^{c} is an exemplary implementation of prosthetic valve assembly 200, and thus includes all of the features described for prosthetic valve assembly 200 throughout the current disclosure, except that prosthetic valve assembly 200^{c} includes a plurality of expansion control rods 250 and at least two flexible tension members 280 that comprise a proximal flexible tension members 280′, positioned closer to outflow end 102, and a distal flexible tension members 280″, positioned closer to inflow end 104. At least one of the expansion control rods 250 is coupled, by a corresponding proximal local loop 282′, to the proximal flexible tension members 280′ but not to the distal flexible tension members 280″, and at least one of the expansion control rods 250 is coupled, by a corresponding distal local loop 282″, to the distal flexible tension members 280″ but not to the proximal flexible tension members 280′.

In the example illustrated in FIG. 5, prosthetic valve assembly 200^{c} is shown to include eight expansion control rods 250, four of which are coupled to the proximal flexible tension members 280′ but not to the distal flexible tension members 280″, and four of which are coupled to the distal flexible tension members 280″ but not to the proximal flexible tension members 280′. The rods 250 attached to the proximal 280′ and distal 280″ flexible tension members can be circumferentially arranged in an alternating manner as illustrated, though any other arrangement is contemplated.

Retrieval of one or more of the rods 250 coupled to the proximal flexible tension members 280′ serves to free the corresponding sections of the proximal flexible tension members 280′, allowing the outflow end portion of the frame 106 to expand to a greater diameter, while retrieval of one or more of the rods 250 coupled to the distal flexible tension members 280″ serves to free the corresponding sections of the distal flexible tension members 280″, allowing the inflow end portion of the frame 106 to expand to a greater diameter. In this manner, any of the inflow and outflow portions of the frame can optionally be allowed to expand to a different diameter Dᵥ by independently removing an appropriate number of rods 250 attached to either of the proximal 280′ and distal 280″ flexion tension members. For example, releasing a greater number of rods 250 coupled to the proximal flexible tension members 280′ can optionally allow the frame 106 to expand to a V-shaped profile, while releasing a greater number of rods 250 coupled to the distal flexible tension members 280″ can optionally allow the frame 106 to expand to an A-shaped profile. If a cylindrical expanded profile is desired, the same number of rods coupled to either one of the proximal 280′ and distal 280″ flexion tension members can be optionally retracted.

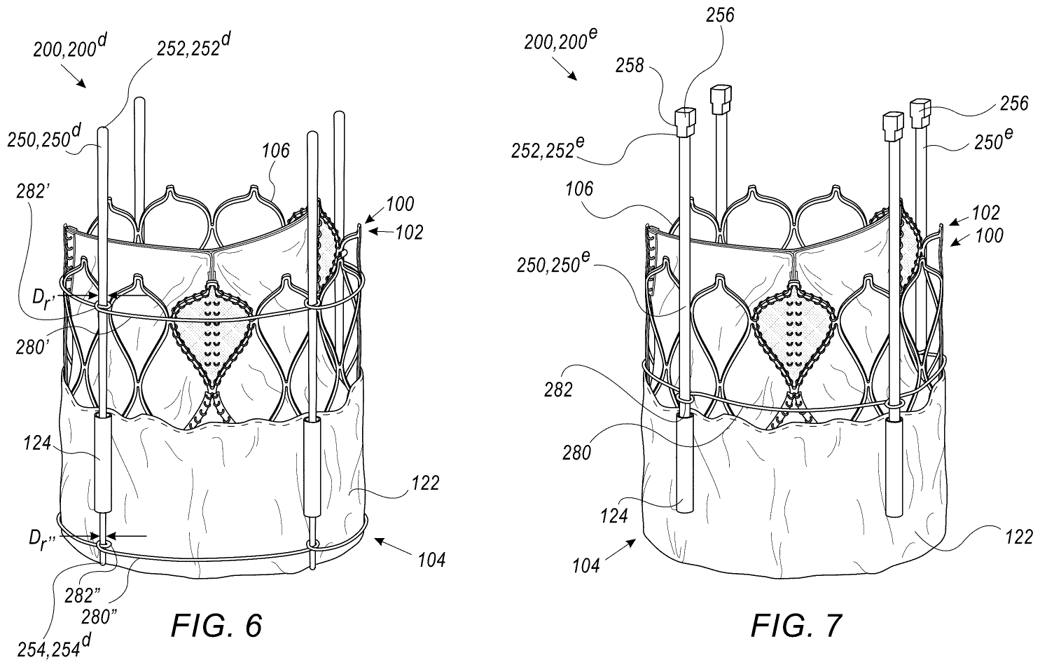

FIG. 6 shows an exemplary prosthetic valve assembly 200^{d}. Prosthetic valve assembly 200^{d} is an exemplary implementation of prosthetic valve assembly 200, and thus includes all of the features described for prosthetic valve assembly 200 throughout the current disclosure, except that prosthetic valve assembly 200^{d} includes at least one expansion control rod 250^{d} having a non-uniform rod diameter Dᵣ, attached both to a proximal flexible tension member 280′ and a distal flexible tension member 280″ in a manner similar to that described above for prosthetic valve assembly 200^{b} with respect to FIG. 4.

In the example illustrated in FIG. 6, each of a plurality of expansion control rods 250^{d} is shaped to define a rod diameter D_{r′} at the level of proximal flexible tension member 280′, and a rod diameter D_{r″} at the level of distal flexible tension member 280″, such that the rod diameter D_{r′} around which local loop 282′ is looped, is greater than rod diameter D_{r″} around which local loop 282″ is looped. In this manner, when rods 250^{d} are retracted, the corresponding freed sections of the distal flexible tension member 280″ contribute less than the freed sections of the proximal flexible tension member 280′, so as to allow the frame 106 to expand to a V-shaped profile. In some examples, rod diameter D_{r′} can optionally be greater than rod diameter D_{r″} (configuration not illustrated), so as to allow the frame 106 to expand to an A-shaped profile.

In some cases, retraction of the sheath or capsule of a delivery apparatus during prosthetic valve 100 implantation may cause the prosthetic valve to move distally or proximally relative to the native valve, and repositioning of the prosthetic valve may be required as it is progressively exposed from the sheath. Such repositioning can become increasingly difficult for the clinician to perform and/or potentially dangerous to the patient as additional sections of the prosthetic valve are exposed from the sheath and expanded. In such cases, it may be desirable to allow similar removal of expansion control rods 250 at the site of implantation, within the patient's body, instead of (or in addition to) rod removal outside the patient's body prior to the implantation procedure.

FIG. 7 shows an exemplary prosthetic valve assembly 200^{e}. Prosthetic valve assembly 200^{e} is an exemplary implementation of prosthetic valve assembly 200, and thus includes all of the features described for prosthetic valve assembly 200 throughout the current disclosure, except that prosthetic valve assembly 200^{e} includes at least one expansion control rod 250^{e} further comprising a rod head 256 at the rod proximal end portion 252^{e}. The rod head 256 can optionally further define one or more shoulders 258 facing distally. As will be further described below, a pull assembly of a delivery apparatus can optionally releasably engage with the head 256 and shoulders 258.

While prosthetic valve assembly 200^{e} is illustrated in FIG. 7 and in FIGS. 9A-9B further described below, to include four expansion control rod 250^{e} coupled to a single flexible tension member 280 that can optionally be positioned approximately halfway between the inflow end 104 and the outflow end 102 of the prosthetic valve 100 in a manner similar to that illustrated and described above for prosthetic valve assembly 200^{a} with respect to FIGS. 2A-2C, this is shown by way of illustration and not limitation, and that other than having expansion control rod 250^{e} further equipped with rod heads 256, prosthetic valve assembly 200^{e} can optionally be arranged in a similar manner to that described for any of prosthetic valve assemblies 200^{b}, 200^{c}, 200^{d} above, such as by including any number of expansion control rod 250^{e}, including more than a one flexible tension member 280 (such as proximal and distal flexible tension members 280′ and 280″), having all or some of the rods 250^{e} coupled either to one, some, or all of the flexible tension members, as well as by forming the remainder of the rods 250^{e} extending distally from rod heads 256 either as uniform-diameter rods (similar to expansion control rod 250^{a}, for example) or tapering rods (similar to expansion control rod 250^{d}, for example).

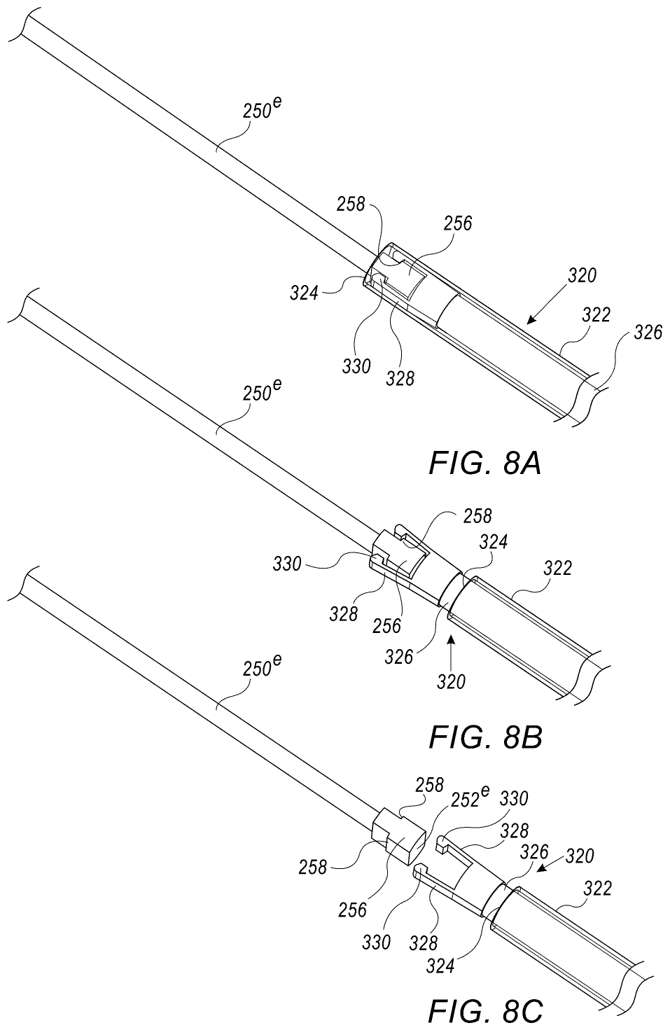

FIGS. 8A-8C illustrated a distal portion of a pull-assembly 320 of an exemplary delivery apparatus 302 (also indicated in FIGS. 9A-9B), shown engaged with a proximal portion of an expansion control rod 250^{e} (FIG. 8A) and in steps of disengagement therefrom (FIGS. 8B-8C). A delivery apparatus 302 can optionally include one or more pull-assemblies 320, matching the number of expansion control rod 250^{e}. Each pull-assembly 320 can optionally include an outer sleeve 322 and a releasable pull member 326 extending through the outer sleeve 322. In the illustrated example, the releasable pull member 326 includes one or more flexible elongated members 328 that can optionally have radial protrusions 330 configured to engage the shoulders 258 of the rod head 256 as shown in FIG. 8A.

As shown in FIG. 8A, when the pull-assembly 320 is coupled to expansion control rod 250^{e}, the releasable pull member 326 is aligned with the rod head 256 and the flexible elongated members 328 are positioned extend along the sides of the rod head 256. The outer sleeve is disposed over the distal portion of the releasable pull member 326 to radially compress the flexible elongated members 328 against the rod head 256 such that the radial protrusions 330 abut the shoulders 258, thereby coupling the releasable pull member 326 to the expansion control rod 250^{e}. The outer sleeve 322 can optionally be positioned in this state such that a distal lip 324 thereof is positioned distal to the rod head 256 and/or distal to the radial protrusions 330.

The outer sleeve 322 is axially movable with respect to the releasable pull member 326. As shown in FIG. 8B, axially pulling the outer sleeve 322 relative to the releasable pull members 326, for example such that the distal lip 324 of the outer sleeve 322 is proximal to the flexible elongated members 328, allows the proximal to the flexible elongated members 328 to spring away from the rod head 256. As shown, flexible elongated members 328 are configured to be biased radially outward to an expanded state, for example, by shape setting the members 328. Optionally, the releasable pull member 326 can comprise a laser-cut Nitinol tube, which can be heat-set such that the flexible elongated members 328 bias radially outwardly. Thus, when the outer sleeve 322 is proximally pulled, the flexible elongated members 328 are biased away from the rod head 256 such that the radial protrusions 330 are spaced away from, and no longer abut, the shoulders 258.

The releasable pull member can optionally be in the form of an elongated cable, tube or shaft. As shown in FIG. 8C, once the flexible elongated members 328 are no longer engaged with rod head 256, the pull-assembly 320, including the releasable pull member 326 and outer sleeve 322, can optionally be further puled proximally away from the expansion control rod 250^{e}.

The delivery apparatus 302 can optionally include a handle (not shown) equipped with one or more knobs configured to perform various functions of the delivery apparatus 302. For example, a knob of the handle can optionally be configured to release one or more pull-assemblies 320 from the corresponding expansion control rods 250^{e}. Such a knob can optionally operate a mechanism configured to axially pull an outer sleeve 322 as shown in FIG. 8B. The mechanism can optionally be further configured to pull both the releasable pull member 326 and the outer sleeve 322 to retract the pull-assembly 320 as shown in FIG. 8C, following initial pull of the outer sleeve shown in FIG. 8B.

FIGS. 9A-9B show a distal portion of an exemplary delivery assembly 300 in different states of engagement with prosthetic valve 100. Delivery assembly 300 comprises a delivery apparatus 302 and a prosthetic valve assembly 200^{e}, wherein the prosthetic valve assembly 200^{e} comprises at least one expansion control rod 250^{e} releasably coupled to prosthetic valve 100, and wherein the delivery apparatus 302 comprises at least one pull-assembly 320 releasably coupled to the at least one expansion control rod 250^{e}. The delivery apparatus 302 can optionally include, in some examples, an outer shaft 308 extending from a handle (not shown) of the delivery apparatus 302. Optionally, a capsule (not separately illustrated) can be attached to a distal end of the outer shaft 308, though this is not mandatory and a distal portion of the outer shaft 308 can optionally serve as a capsule for prosthetic valve 100.

After coupling the expansion control rod(s) 250 to the prosthetic valve 100 in an expanded state thereof, as described above, the prosthetic valve 100 can optionally be crimped or compressed and placed into a distal portion of the delivery shaft 310 or a capsule attached thereto, and delivered in this state, through the patient's vasculature. Upon reaching the site of implantation, the outer shaft 308 can optionally be proximally retracted from the prosthetic valve assembly 200, exposing the prosthetic valve 100 and expansion control rod(s) 250, allowing the valve to self-expand as shown in FIG. 9A. In the state illustrated in FIG. 9A, self-expansion of the valve 100 is restricted by the at least one flexible tension member 280, which can optionally be coupled, at this stage, to one or more expansion control rod(s) 250^{e}, and optionally to all of the expansion control rods 250^{e}, such as all four control rods in the illustrated example.

In some examples, the delivery apparatus 302 further comprises an optional delivery shaft 310 extending through a lumen of the outer shaft 308. The pull-assemblies 320 can optionally extend through delivery shaft 310. In some examples, the outer shaft 308 can optionally be a multi-lumen shaft, such that each of a plurality of pull-assemblies 320 can optionally extend through a separate lumen. While not illustrated, a delivery apparatus can optionally further include a nosecone shaft extending through delivery shaft 310, carrying a nosecone (not shown) attached to a distal end thereof, and through which a guidewire (not shown) can be optionally passed. The outer shaft 308 and the delivery shaft 310 can optionally be axially movable relative to each other, such that deployment of prosthetic valve 100 as shown in FIG. 9A can be achieve by proximally pulling outer shaft 308 relative to delivery shaft 310, and/or distally pushing delivery shaft 310 relative to outer shaft 308.

During delivery through the patient's vasculature and deployment of the prosthetic valve 100 as shown in FIG. 9A, the pull-assemblies 320 remain coupled to the expansion control rods 250^{e}, as described an illustrated for example with respect to FIG. 8A, such that initial self-expansion of the valve 100 is restricted, for example to a minimal diameter of a range of working diameters as described above. In some examples, the expansion control rods 250^{e} of prosthetic valve assembly 200^{e} can optionally be arranged to restrict self-expansion of the valve 100 to a partially compressed diameter which can be less than the working diameter, upon release from the outer shaft 308 or a capsule attached thereto, such that a position of the prosthetic valve 100 can optionally be adjusted by the clinician after it is free of the outer shaft 308 or capsule (unlike conventional self-expanding valve delivery).

As shown in FIG. 9B, when the prosthetic valve 100 is in a desired position, a selected number of pull-assemblies 320 can optionally be released from the respective expansion control rods 250^{e}, while one or more of the remaining pull-assemblies 320 can optionally preserve engagement with the corresponding remaining expansion control rods 250^{e}. Once a desired number of pull-assemblies 320 are released from the respective expansion control rods 250^{e}, the entire delivery apparatus 302 can optionally be retracted from prosthetic valve 100, such that any of the expansion control rod(s) 250^{e} disengaged from pull-assemblies 320 remain coupled to the prosthetic valve 100, while any expansion control rod(s) 250^{e} that remain coupled to pull-assemblies 320 are proximally pulled therewith during retraction of the delivery apparatus 302 from the valve 100.

In the example illustrated in FIG. 9B, three pull-assemblies 320b, 320c and 320d are disengaged from expansion control rods 250^{e}b, 250^{e}c and 250^{e}d, respectively, while pull-assembly 320a remains engaged with expansion control rod 250^{e}a. As shown in FIG. 9B, this results, during retraction of the delivery apparatus 302 and the pull assemblies 320 from valve 100, release of the expansion control rod 250^{e}a from prosthetic valve 100, freeing the section that formed local loop 282a prior to this release, allowing further partial expansion of the valve 100, while the remaining expansion control rods 250^{e}b, 250^{e}C and 250^{e}d remain coupled to prosthetic valve 100 with local loops 282b, 282c and 282d looped therearound, in a manner similar to that illustrated and described above with respect to FIG. 2B for example.

While examples of a prosthetic valve assemblies 200 described above are shown to include an exemplary prosthetic valve that includes a valvular structure 130, it should be understood that a prosthetic valve assembly according to any example of the current disclosure can optionally be used for implantation of other prosthetic devices aside from prosthetic valves, such as stents or grafts.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve assembly comprising: a prosthetic valve comprising an annular frame movable between a radially compressed and a radially expanded configuration and defining a central longitudinal axis; at least one expansion control rod disposed radially outward to the frame, extending between a rod proximal end portion and a rod distal end portion in parallel to the central longitudinal axis; and at least one flexible tension member extending circumferentially around the frame, and comprising at least one local loop wrapped around the at least one expansion control rod; wherein the at least one flexible tension member is configured to radially expand to a first diameter when the at least one local loop is wrapped around the at least one expansion control rod, and expand to a second diameter greater than the first diameter, when the at least one expansion control rod is retracted from the at least one flexible tension member, thereby allowing the corresponding at least one local loop to unravel.

Example 2. The prosthetic valve assembly of any example herein, particularly example 1, wherein the frame is a self-expandable frame.

Example 3. The prosthetic valve assembly of any example herein, particularly example 1 or example 2, wherein the rod proximal end is proximal to the frame.

Example 4. The prosthetic valve assembly of any example herein, particularly any one of examples 1 to 3, wherein the at least one flexible tension member comprises at least one of: a wire, a string, a suture, and/or a cable.

Example 5. The prosthetic valve assembly of any example herein, particularly any one of examples 1 to 4, wherein the prosthetic valve further comprises an outer skirt disposed around the frame, wherein the outer skirt comprises at least one axially extending sleeve configured to accommodate at least a portion of the corresponding at least one expansion control rod.

Example 6. The prosthetic valve assembly of any example herein, particularly any one of examples 1 to 5, wherein the at least one expansion control rod comprises a plurality of expansion control rods, wherein the at least one flexible tension member is configured to radially expand to the first diameter when all of the expansion control rods are coupled thereto, to radially expand to the second diameter when a first expansion control rod of the plurality of control rods is retracted therefrom, and to radially expand to a third diameter which is greater than the second diameter, when a second expansion control rod of the plurality of control rods is retracted therefrom.

Example 7. The prosthetic valve assembly of any example herein, particularly example 6, wherein the expansion control rods are equally spaced from each other around the circumference of the frame.

Example 8. The prosthetic valve assembly of any example herein, particularly any one of examples 1 to 7, wherein the at least one flexible tension member comprises a single flexible tension member.

Example 9. The prosthetic valve assembly of any example herein, particularly example 8, wherein the flexible tension member is equally spaced between an inflow end and an outflow end of the frame.

Example 10. The prosthetic valve assembly of any example herein, particularly any one of examples 1 to 7, wherein the at least one flexible tension member comprises a plurality of flexible tension members axially spaced from each other.

Example 11. The prosthetic valve assembly of any example herein, particularly any one of examples 1 to 5, wherein the at least one expansion control rod comprises a first expansion control rod and a second expansion control rod, wherein the at least one flexible tension member comprises a proximal flexible tension member and a distal flexible tension member, wherein the first expansion control rod is coupled to a corresponding local loop of the proximal flexible tension member but not to the distal flexible tension member, and wherein the second expansion control rod is coupled to a corresponding local loop of the distal flexible tension member but not to the proximal flexible tension member.

Example 12. The prosthetic valve assembly of any example herein, particularly any one of examples 1 to 7, wherein the at least one flexible tension member comprises a proximal flexible tension member and a distal flexible tension member, wherein the at least one expansion control rod defines a non-uniform rod diameter along a length thereof, and wherein the at least one expansion control rod is coupled to respective local loops of both the proximal flexible tension member and the distal flexible tension member.

Example 13. The prosthetic valve assembly of any example herein, particularly example 12, wherein the rod diameter at the level of the proximal flexible tension member is greater than the rod diameter at the level of the distal flexible tension member.

Example 14. The prosthetic valve assembly of any example herein, particularly example 12, wherein the rod diameter at the level of the distal flexible tension member is greater than the rod diameter at the level of the proximal flexible tension member.

Example 15. The prosthetic valve assembly of any example herein, particularly any one of examples 1 to 14, wherein the at least one expansion control rod comprises a rod head at the rod proximal end portion, the rod head defining at least one shoulder.

Example 16. The prosthetic valve assembly of any example herein, particularly any one of examples 1 to 15, wherein the at least one flexible tension member is coupled to the frame via one or more sutures.

Example 17. The prosthetic valve assembly of any example herein, particularly any one of examples 1 to 15, wherein the at least one flexible tension member is coupled to the frame by weaving the at least one flexible tension member along struts of the frame in an in-and-out pattern.

Example 18. The prosthetic valve assembly of any example herein, particularly any one of examples 1 to 17, wherein the prosthetic valve further comprises a valvular structure comprising a plurality of leaflets disposed inside the frame and configured to regulate flow of blood through the prosthetic valve.

Example 19. The prosthetic valve assembly of any example herein, particularly example 18, wherein the plurality of leaflets comprises three leaflets.

Example 20. A method of assembling a prosthetic valve assembly, comprising: providing a prosthetic valve that includes a frame movable between a radially compressed and a radially expanded configuration, in a radially expanded configuration of the frame; positioning at least one expansion control rod radially outward of the frame; extending at least one flexible tension member around the frame; and forming at least one local loop by wrapping the at least one flexible tension member around the at least one expansion control rod.

Example 21. The method of any example herein, particularly example 20, wherein the forming the at least one local loop is performed prior to the positioning the at least one expansion control rod and the extending the at least one flexible tension member.

Example 22. The method of any example herein, particularly example 21, wherein the positioning the at least one expansion control rod and the extending the at least one flexible tension member are performed simultaneously.

Example 23. The method of any example herein, particularly example 20, wherein the extending the at least one flexible tension member if performed prior to the positioning the at least one expansion control rod.

Example 24. The method of any example herein, particularly any one of examples 20 to 23, further comprising at least partially compressing the frame prior to the positioning the at least one expansion control rod.

Example 25. The method of any example herein, particularly any one of examples 20 to 23, further comprising at least partially compressing the frame prior to the extending the at least one flexible tension member.

Example 26. The method of any example herein, particularly any one of examples 20 to 25, wherein the frame is a self-expandable frame.

Example 27. The method of any example herein, particularly any one of examples 20 to 26, wherein the at least one flexible tension member comprises at least one of: a wire, a string, a suture, and/or a cable.

Example 28. The method of any example herein, particularly any one of examples 20 to 27, wherein the positioning at least one expansion control rod comprises inserting at least a portion of the at least one expansion control rod into an axially extending sleeve of an outer skirt disposed around the frame.

Example 29. The method of any example herein, particularly any one of examples 20 to 28, wherein the extending at least one flexible tension member comprises coupling the at least one flexible tension member to the frame.

Example 30. The method of any example herein, particularly example 29, wherein the coupling the at least one flexible tension member comprises suturing the at least one flexible tension member to the frame.

Example 31. The method of any example herein, particularly example 29, wherein the coupling the at least one flexible tension member comprises weaving the at least one flexible tension member along struts of the frame in an in-and-out pattern.

Example 32. The method of any example herein, particularly any one of examples 20 to 31, wherein the at least one flexible tension member comprises a proximal flexible tension member and a distal flexible tension member, and wherein the extending at least one flexible tension member comprises positioning the proximal flexible tension member closer to an outflow end of the frame than an inflow end thereof, and positioning the distal flexible tension member closer to the inflow end of the frame than to its outflow end.

Example 33. The method of any example herein, particularly example 32, wherein the forming the at least one local loop comprises forming at least one local loop of the proximal flexible tension member around the at least one expansion control rod, and forming at least one local loop of the distal flexible tension member around the same expansion control rod.

Example 34. The method of any example herein, particularly example 33, wherein the at least one expansion control rod defines a non-uniform rod diameter along a length thereof.

Example 35. The method of any example herein, particularly example 32, wherein the at least one expansion control rod comprises a plurality of expansion control rods, and wherein forming the at least one local loop comprises forming a local loop of the proximal flexible tension member around a first expansion control rod of the plurality of expansion rods, but not around a second expansion control rod, and forming a local loop of the distal flexible tension member around the second expansion control rod, but not around the first expansion control rod.

Example 36. The method of any example herein, particularly any one of examples 20 to 35, further comprising crimping the prosthetic valve to the radially compressed configuration of the frame.

Example 37. The method of any example herein, particularly example 36, further comprising inserting the prosthetic valve, in the radially compressed configuration, into a capsule of a delivery apparatus.

Example 38. A method comprising: selecting a maximum diameter for a prosthetic valve assembly based at least in part on a patient's native anatomy, the prosthetic valve assembly comprising a prosthetic valve comprising an annular frame movable between a radially compressed and a radially expanded configuration, at least one expansion control rod disposed radially outward to the frame, and at least one flexible tension member extending circumferentially around the frame, wherein the at least one expansion control rod is coupled to the at least one flexible tension member via a local loop of the at least one flexible tension member wrapped around the at least one expansion control rod; retracting any of the at least one expansion control rod from the at least one flexible tension member in a manner that unravels the corresponding local loop, so as to allow the at least one flexible tension member to expand to the maximum diameter; crimping the prosthetic valve to the radially compressed configuration of the frame; inserting the crimped prosthetic valve into a capsule of a delivery apparatus; advancing the delivery assembly to a selected implantation site inside the body of the patient; and deploying the prosthetic valve assembly out of the capsule, thereby allowing it to expand to the selected maximum diameter.

Example 39. The method of any example herein, particularly example 38, wherein the frame is a self-expandable frame.

Example 40. The method of any example herein, particularly example 38 or example 39, wherein the at least one flexible tension member comprises at least one of: a wire, a string, a suture, and/or a cable.

Example 41. The method of any example herein, particularly any one of examples 38 to 40, wherein the at least one expansion control rod comprises a plurality of expansion control rods, and wherein the retracting any of the at least one expansion control rod comprises retracting one or more of the plurality of expansion control rods.

Example 42. The method of any example herein, particularly any one of examples 38 to 40, wherein the at least one flexible tension member comprises a proximal flexible tension member and a distal flexible tension member.

Example 43. The method of any example herein, particularly example 42, wherein the selecting a maximum diameter comprises selecting a maximum diameter of an inflow end of the frame and selecting a maximum diameter of an outflow end of the frame.

Example 44. The method of any example herein, particularly example 43, wherein the at least one expansion control rod defines a non-uniform rod diameter along a length thereof, such that the rod diameter at the level of the proximal flexible tension member is different than the rod diameter at the level of the distal flexible tension member.

Example 45. The method of any example herein, particularly example 43, wherein the at least one expansion control rod comprises a plurality of expansion control rods, the plurality of expansion control rods comprising a first expansion control rod coupled to a corresponding local loop of the proximal flexible tension member but not to the distal flexible tension member, and a second expansion control rod coupled to a corresponding local loop of the distal flexible tension member but not to the proximal flexible tension member.

Example 46. The method of any example herein, particularly any one of examples 38 to 46, wherein the patient's native anatomy is determined using at least one of an angiogram and a CT-scan.

Example 47. A delivery assembly comprising: a prosthetic valve assembly comprising: a prosthetic valve comprising an annular frame movable between a radially compressed and a radially expanded configuration and defining a central longitudinal axis; at least one expansion control rod disposed radially outward to the frame, extending between a rod proximal end portion and a rod distal end portion in parallel to the central longitudinal axis; and at least one flexible tension member extending circumferentially around the frame, and comprising at least one local loop wrapped around the at least one expansion control rod; and a delivery apparatus comprising at least one pull-assembly that comprises: a releasable pull member releasably coupled to the at least one expansion control rod; and an outer sleeve disposed around, and axially movable relative to, the releasable pull member; wherein the at least one pull-assembly is configured to release the at least one expansion control rod from the at least one flexible tension member by axially pulling it when coupled thereto; and wherein the at least one flexible tension member is configured to radially expand to a first diameter when the at least one local loop is wrapped around the at least one expansion control rod, and expand to a second diameter greater than the first diameter, when the at least one expansion control rod is retracted from the at least one flexible tension member by the at least one pull-assembly, thereby allowing the corresponding at least one local loop to unravel.

Example 48. The delivery assembly of any example herein, particularly example 47, wherein the frame is a self-expandable frame.

Example 49. The delivery assembly of any example herein, particularly example 47 or example 48, wherein the at least one expansion control rod comprises a rod head at the rod proximal end portion, the rod head defining at least one shoulder extending laterally relative to the longitudinal axis of the expansion control rod.

Example 50. The delivery assembly of any example herein, particularly example 49, wherein the releasable pull member comprises at least one flexible elongated member releasably coupled to the at least one shoulder.

Example 51. The delivery assembly of any example herein, particularly example 50, wherein the at least one flexible elongated member comprises a radial protrusion configured to abut the at least one shoulder.

Example 52. The delivery assembly of any example herein, particularly example 50 or example 51, wherein the at least one flexible elongated member is biased radially outwardly from the rod head.

Example 53. The delivery assembly of any example herein, particularly any one of examples 49 to 52, wherein the rod head is proximal to the frame.

Example 54. The delivery assembly of any example herein, particularly any one of examples 47 to 53, wherein the at least one flexible tension member comprises at least one of: a wire, a string, a suture, and/or a cable.

Example 55. The delivery assembly of any example herein, particularly any one of examples 47 to 54, wherein the prosthetic valve further comprises an outer skirt disposed around the frame, wherein the outer skirt comprises at least one axially extending sleeve configured to accommodate at least a portion of the corresponding at least one expansion control rod.

Example 56. The delivery assembly of any example herein, particularly any one of examples 47 to 55, wherein the at least one expansion control rod comprises a plurality of expansion control rods, wherein the at least one pull-assembly comprises a plurality of pull-assemblies, and wherein the number of pull assemblies matches the number of expansion control rods.

Example 57. The delivery assembly of any example herein, particularly example 56, wherein the at least one flexible tension member is configured to radially expand to the first diameter when all of the expansion control rods are coupled thereto, to radially expand to the second diameter when a first expansion control rod of the plurality of control rods is retracted therefrom by a first pull assembly of the plurality of pull-assemblies, and to radially expand to a third diameter which is greater than the second diameter, when a second expansion control rod of the plurality of control rods is retracted therefrom by a second pull-assembly of the plurality of pull assemblies.

Example 58. The delivery assembly of any example herein, particularly example 56, wherein the at least one flexible tension member comprises a proximal flexible tension member and a distal flexible tension member, wherein a first expansion control rod of the plurality of expansion control rods is coupled to a corresponding local loop of the proximal flexible tension member but not to the distal flexible tension member, and wherein a second expansion control rod of the plurality of expansion control rods is coupled to a corresponding local loop of the distal flexible tension member but not to the proximal flexible tension member.

Example 59. The delivery assembly of any example herein, particularly any one of examples 47 to 55, wherein the at least one flexible tension member comprises a proximal flexible tension member and a distal flexible tension member, wherein the at least one expansion control rod defines a non-uniform rod diameter along a length thereof, and wherein the at least one expansion control rod is coupled to respective local loops of both the proximal flexible tension member and the distal flexible tension member.

Example 60. The delivery assembly of any example herein, particularly example 59, wherein the rod diameter at the level of the proximal flexible tension member is greater than the rod diameter at the level of the distal flexible tension member.

Example 61. The delivery assembly of any example herein, particularly example 59, wherein the rod diameter at the level of the distal flexible tension member is greater than the rod diameter at the level of the proximal flexible tension member.

Example 62. The delivery assembly of any example herein, particularly any one of examples 47 to 61, wherein the prosthetic valve further comprises a valvular structure comprising a plurality of leaflets disposed inside the frame and configured to regulate flow of blood through the prosthetic valve.

Example 63. The delivery assembly of any example herein, particularly example 62, wherein the plurality of leaflets comprises three leaflets.

Example 64. A prosthetic valve assembly comprising: a prosthetic valve comprising an annular frame movable between a radially compressed and a radially expanded configuration; at least one expansion control rod disposed radially outward to the frame; and at least one flexible tension member extending circumferentially around the frame and comprising at least one local loop wrapped around the at least one expansion control rod.

Example 65. A delivery assembly comprising: a prosthetic valve assembly comprising: a prosthetic valve comprising an annular frame movable between a radially compressed and a radially expanded configuration; at least one expansion control rod disposed radially outward to the frame; and at least one flexible tension member extending circumferentially around the frame and comprising at least one local loop wrapped around the at least one expansion control rod; and a delivery apparatus comprising: a releasable pull member releasably coupled to the at least one expansion control rod; and an outer sleeve disposed around, and axially movable relative to, the releasable pull member.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve assembly comprising:
a prosthetic valve comprising an annular frame movable between a radially compressed and a radially expanded configuration and defining a central longitudinal axis;
at least one expansion control rod disposed radially outward to the frame, extending between a rod proximal end portion and a rod distal end portion in parallel to the central longitudinal axis; and
at least one flexible tension member extending circumferentially around the frame, and comprising at least one local loop wrapped around the at least one expansion control rod;
wherein the at least one flexible tension member is configured to radially expand to a first diameter when the at least one local loop is wrapped around the at least one expansion control rod, and expand to a second diameter greater than the first diameter, when the at least one expansion control rod is retracted from the at least one flexible tension member, thereby allowing the corresponding at least one local loop to unravel.

2. The prosthetic valve assembly of claim 1, wherein the frame is a self-expandable frame.

3. The prosthetic valve assembly of claim 1, wherein the prosthetic valve further comprises an outer skirt disposed around the frame, wherein the outer skirt comprises at least one axially extending sleeve configured to accommodate at least a portion of the corresponding at least one expansion control rod.

4. The prosthetic valve assembly of claim 1, wherein the at least one expansion control rod comprises a plurality of expansion control rods, wherein the at least one flexible tension member is configured to radially expand to the first diameter when all of the expansion control rods are coupled thereto, to radially expand to the second diameter when a first expansion control rod of the plurality of control rods is retracted therefrom, and to radially expand to a third diameter which is greater than the second diameter, when a second expansion control rod of the plurality of control rods is retracted therefrom.

5. The prosthetic valve assembly of claim 1, wherein the at least one flexible tension member comprises a plurality of flexible tension members axially spaced from each other.

6. The prosthetic valve assembly of claim 1, wherein the at least one expansion control rod comprises a first expansion control rod and a second expansion control rod, wherein the at least one flexible tension member comprises a proximal flexible tension member and a distal flexible tension member, wherein the first expansion control rod is coupled to a corresponding local loop of the proximal flexible tension member but not to the distal flexible tension member, and wherein the second expansion control rod is coupled to a corresponding local loop of the distal flexible tension member but not to the proximal flexible tension member.

7. The prosthetic valve assembly of claim 1, wherein the at least one flexible tension member comprises a proximal flexible tension member and a distal flexible tension member, wherein the at least one expansion control rod defines a non-uniform rod diameter along a length thereof, and wherein the at least one expansion control rod is coupled to respective local loops of both the proximal flexible tension member and the distal flexible tension member.

8. The prosthetic valve assembly of claim 1, wherein the at least one flexible tension member is coupled to the frame via one or more sutures.

9. The prosthetic valve assembly of claim 1, wherein the at least one flexible tension member is coupled to the frame by weaving the at least one flexible tension member along struts of the frame in an in-and-out pattern.

10. A method comprising:
selecting a maximum diameter for a prosthetic valve assembly based at least in part on a patient's native anatomy, the prosthetic valve assembly comprising a prosthetic valve comprising an annular frame movable between a radially compressed and a radially expanded configuration, at least one expansion control rod disposed radially outward to the frame, and at least one flexible tension member extending circumferentially around the frame, wherein the at least one expansion control rod is coupled to the at least one flexible tension member via a local loop of the at least one flexible tension member wrapped around the at least one expansion control rod;
retracting any of the at least one expansion control rod from the at least one flexible tension member in a manner that unravels the corresponding local loop, so as to allow the at least one flexible tension member to expand to the maximum diameter;
crimping the prosthetic valve to the radially compressed configuration of the frame;
inserting the crimped prosthetic valve into a capsule of a delivery apparatus;
advancing the delivery assembly to a selected implantation site inside the body of the patient; and
deploying the prosthetic valve assembly out of the capsule, thereby allowing it to expand to the selected maximum diameter.

11. The method of claim 10, wherein the at least one expansion control rod comprises a plurality of expansion control rods, and wherein the retracting any of the at least one expansion control rod comprises retracting one or more of the plurality of expansion control rods.

12. The method of claim 10, wherein the at least one flexible tension member comprises a proximal flexible tension member and a distal flexible tension member.

13. The method of claim 12, wherein the selecting a maximum diameter comprises selecting a maximum diameter of an inflow end of the frame and selecting a maximum diameter of an outflow end of the frame.

14. The method of claim 13, wherein the at least one expansion control rod comprises a plurality of expansion control rods, the plurality of expansion control rods comprising a first expansion control rod coupled to a corresponding local loop of the proximal flexible tension member but not to the distal flexible tension member, and a second expansion control rod coupled to a corresponding local loop of the distal flexible tension member but not to the proximal flexible tension member.

15. A delivery assembly comprising:
a prosthetic valve assembly comprising:
a prosthetic valve comprising an annular frame movable between a radially compressed and a radially expanded configuration and defining a central longitudinal axis;
at least one expansion control rod disposed radially outward to the frame, extending between a rod proximal end portion and a rod distal end portion in parallel to the central longitudinal axis; and
at least one flexible tension member extending circumferentially around the frame, and comprising at least one local loop wrapped around the at least one expansion control rod; and
a delivery apparatus comprising at least one pull-assembly that comprises:
a releasable pull member releasably coupled to the at least one expansion control rod; and
an outer sleeve disposed around, and axially movable relative to, the releasable pull member;
wherein the at least one pull-assembly is configured to release the at least one expansion control rod from the at least one flexible tension member by axially pulling it when coupled thereto; and
wherein the at least one flexible tension member is configured to radially expand to a first diameter when the at least one local loop is wrapped around the at least one expansion control rod, and expand to a second diameter greater than the first diameter, when the at least one expansion control rod is retracted from the at least one flexible tension member by the at least one pull-assembly, thereby allowing the corresponding at least one local loop to unravel.

16. The delivery assembly of claim 15, wherein the at least one expansion control rod comprises a rod head at the rod proximal end portion, the rod head defining at least one shoulder extending laterally relative to the longitudinal axis of the expansion control rod.

17. The delivery assembly of claim 16, wherein the releasable pull member comprises at least one flexible elongated member releasably coupled to the at least one shoulder.

18. The delivery assembly of claim 17, wherein the at least one flexible elongated member comprises a radial protrusion configured to abut the at least one shoulder.

19. The delivery assembly of claim 17, wherein the at least one flexible elongated member is biased radially outwardly from the rod head.

20. The delivery assembly of claim 15, wherein the at least one expansion control rod comprises a plurality of expansion control rods, wherein the at least one pull-assembly comprises a plurality of pull-assemblies, and wherein the number of pull assemblies matches the number of expansion control rods.
